(19) United States
(12) Patent Application Publication
(10) Pub. No.: US 2023/0355152 A1
(43) Pub. Date: Nov. 9, 2023

(54) Bioelectrode

(71) Applicant: SEKISUI POLYMATECH CO., LTD., Saitama (JP)

(72) Inventors: Masamichi Ishikubo, Saitama (JP); Hiroshi Ogasawara, Saitama (JP)

(73) Assignee: SEKISUI POLYMATECH CO., LTD., Saitama (JP)

(21) Appl. No.: 18/003,594

(22) PCT Filed: Jul. 1, 2021
(86) PCT No.: PCT/JP2021/024961

§ 371 Date: Dec. 28, 2022

(30) Foreign Application Priority Data
Jul. 31, 2020 (JP) 2020-130515

(51) Int. Cl. A61B 5/268; A61B 5/00; A61B 5/26; A61B 5/282; B62D 1/06

(52) U.S. Cl. CPC A61B 5/268; A61B 5/6893; A61B 5/26; A61B 5/282; B62D 1/06

(57) ABSTRACT

In a bioelectrode capable of detecting biometric information of a living body in touch with the bioelectrode, the bioelectrode includes a base, a first conductive layer that is laminated on a surface side of the base, that is formed by dispersing scale-shaped conductive particles in an insulating binder, and that has extensibility, and a second conductive layer that is laminated on a surface side of the first conductive layer, that has conductivity, and that is harder than the first conductive layer. The second conductive layer is disposed to be exposed at the surface side of the base where the second conductive layer is touchable with the living body. An amount of conductive particles filled in the second conductive layer is smaller than an amount of the conductive particles filled in the first conductive layer. The second conductive layer has a larger outer contour than the first conductive layer.

### TECHNICAL FIELD

The present invention relates to a bioelectrode.

### BACKGROUND ART

Recently, technical development of a bioelectrode or the like for detecting biometric information, such as a heart rate and an electrocardiogram of an automobile driver, in the form of electrical signals has been progressed to know health conditions and so on of the driver while driving an automobile. As an example of the related art for detecting the biometric information in the form of electrical signals as described above, Patent Literature (PTL) 1 discloses a steering wheel of which surface is covered by a surfacing member that includes, on a surface of a base layer, an elastic layer containing a conductive material. On the other hand, PTL 2 discloses a biometric-information detection steering wheel including a penetrating conductive portion that penetrates through part of a surfacing layer covering a surface of a conductive layer and that is electrically conducted to the conductive layer. Moreover, PTL 3 discloses a biometric information detection device in which a biometric information detection element, such as an electrode, for detecting biometric information of a driver is disposed on a steering wheel.

### CITATION LIST

### Patent Literatures

PTL 1: Japanese Unexamined Patent Application Publication No. 2019-202446
PTL 2: Japanese Unexamined Patent Application Publication No. 2012-157603
PTL 3: WO Publication No. 2004/089209

### SUMMARY OF INVENTION

### Technical Problem

However, because a hand-grasping component, such as a steering wheel grasped by a driver, is directly grasped by hands of the driver, a bioelectrode attached to a surface side of the hand-grasping component tends to wear. This implies the necessity of suppressing aged deterioration caused by wear of the bioelectrode.

The present invention has been made in consideration of the above-described problem and is aimed to improve durability of a bioelectrode.

### Solution to Problem

An embodiment of the present invention provides a bioelectrode capable of detecting biometric information of a living body in touch with the bioelectrode, the bioelectrode including a base, a first conductive layer that is laminated on a surface side of the base, that is formed by dispersing scale-shaped conductive particles in an insulating binder, and that has extensibility, and a second conductive layer that is laminated on a surface side of the first conductive layer, that has conductivity, and that is harder than the first conductive layer, wherein the second conductive layer is disposed to be exposed at the surface side of the base where the second conductive layer is touchable with the living body.

According to the embodiment of the present invention, since the second conductive layer suppresses falling-out of the scale-shaped conductive particles in the first conductive layer laminated on the base and having extensibility, durability of the bioelectrode can be improved.

In an embodiment of the present invention, the second conductive layer may include lump-shaped conductive particles, and an amount of the conductive particles filled in the second conductive layer may be smaller than an amount of the conductive particles filled in the first conductive layer. With that feature, high conductivity can be ensured while falling-out of the scale-shaped conductive particles in the first conductive layer is suppressed.

In an embodiment of the present invention, the second conductive layer may be made of a conductive polymer. With that feature, high conductivity can be ensured while the falling-out of the scale-shaped conductive particles in the first conductive layer is suppressed.

In an embodiment of the present invention, the second conductive layer may have a larger outer contour than the first conductive layer. With that feature, since a surface of the first conductive layer is reliably covered by the second conductive layer, wear of the first conductive layer caused by the falling-out of the scale-shaped conductive particles in the first conductive layer is easier to suppress.

In an embodiment of the present invention, the second conductive layer may be disposed to cover a surface of the first conductive layer. With that feature, wear of the first conductive layer caused by the falling-out of the scale-shaped conductive particles in the first conductive layer can be suppressed.

In an embodiment of the present invention, the second conductive layer may have the same color tone as the base. With that feature, the base can present a more satisfactory external appearance design.

In an embodiment of the present invention, a thickness of the first conductive layer is at least 100 μm or less, and a thickness of the second conductive layer is at least 70 m or less. With that feature, the durability of the bioelectrode can be increased while the conductivity of the bioelectrode is maintained.

In an embodiment of the present invention, the bioelectrode may further include an insulating underlayer between the base and the first conductive layer. With that feature, even when the base is uneven, it is easier to coat the first conductive layer.

Another embodiment of the present invention provides a bioelectrode-equipped steering-wheel surfacing member including one or more bioelectrodes according to any one of the embodiments described above, the one or more bioelectrodes being disposed on a steering wheel surfacing member, or provides a bioelectrode-equipped vehicle interior component including one or more bioelectrodes according to any one of the embodiments described above, the one or more bioelectrodes being disposed on a vehicle interior component.

According to the other embodiment of the present invention, since the one or more bioelectrode according to any one of the embodiments described above are applied to the steering wheel surfacing member or the vehicle interior component, the bioelectrode-equipped steering wheel surfacing member or the bioelectrode-equipped vehicle interior component can be obtained in each of which the durability of the bioelectrodes is improved.

Still another embodiment of the present invention provides a cardiac potential measuring system for detecting, as biometric information of a driver driving a vehicle, a cardiac potential in the form of an electrical signal, the cardiac potential measuring system including a plurality of the bioelectrodes according to any one of the embodiments described above, the bioelectrodes including a first bioelectrode disposed on a steering device of the vehicle, the steering device being operated by the driver, and a second bioelectrode disposed on the steering device or a vehicle interior component disposed in a compartment of the vehicle.

According to the still other embodiment of the present invention, since the durability of the bioelectrodes is improved, change in the cardiac potential as the biometric information of the vehicle driver can be detected with high accuracy in the form of an electrical signal.

In the still other embodiment of the present invention, the vehicle interior component is at least one of a door inner panel, a center console-side armrest, or a shift lever. With that feature, the change in the cardiac potential can be detected as the biometric information with high accuracy in the form of an electrical signal from a location touched by a hand of the driver.

### Advantageous Effects of Invention

According to the present invention, the durability of the bioelectrode can be improved.

### BRIEF DESCRIPTION OF DRAWINGS

FIG. 1(A) is a sectional view illustrating a schematic structure of a bioelectrode according to an embodiment of the present invention, and FIG. 1(B) is an enlarged view of a portion A in FIG. 1(A).

FIGS. 2(A) to 2(D) are explanatory views illustrating a method of manufacturing the bioelectrode according to the embodiment of the present invention.

FIGS. 3(A) to 3(F) are explanatory views illustrating a method of manufacturing a bioelectrode according to another embodiment of the present invention.

FIGS. 4(A) to 4(D) are explanatory views illustrating a method of manufacturing a bioelectrode according to still another embodiment of the present invention.

FIGS. 5(A) and 5(B) are explanatory views illustrating operation of the bioelectrode according to the embodiment of the present invention.

FIG. 6 is an explanatory view illustrating one example of a cardiac potential measuring system to which the bioelectrode according to the embodiment of the present invention is applied.

FIG. 7 is an explanatory view illustrating another example of the cardiac potential measuring system to which the bioelectrode according to the embodiment of the present invention is applied.

### DESCRIPTION OF EMBODIMENTS

Preferred embodiments of the present invention will be described in detail below. Components common to the following embodiments are denoted by the same reference signs, and duplicate description of those components is omitted in the Description. Furthermore, duplicate description of methods of use, operations, and advantageous effects common to the embodiments are also omitted. When words “first” and “second” are used in the Description and Claims, those words are used merely to distinguish different components and are not purported to indicate a particular order, superiority, and so on. It is to be further noted that the embodiments described below are not intended to unreasonably limit the scope of the present invention stated in Claims, and that all of the features described in the embodiments are not always essential to implement the solutions proposed by the present invention.

Structure of Bioelectrode:

A bioelectrode 100 according to an embodiment has the function of detecting biometric information of a driver (living body) in touch with the bioelectrode. The bioelectrode 100 can be disposed in, for example, a surfacing member (steering wheel surfacing member) wound around a rim portion of a steering wheel (steering device) of an automobile (vehicle). In that case, the bioelectrode 100 can be applied to, for example, an electrocardiogram sensor for detecting the biometric information, such as a cardiac potential of the driver grasping the steering wheel, in the form of an electrical signal.

The steering wheel surfacing member including the bioelectrode 100 is one form of a “bioelectrode-equipped steering-wheel surfacing member” according to one embodiment of the present invention. The bioelectrode 100 can be applied to, in addition to the steering wheel, “vehicle interior components” disposed in an automobile compartment, such as a door inner panel, a center console-side armrest, and a shift lever. The vehicle interior component with the bioelectrode 100 is one form of a “bioelectrode-equipped vehicle interior component” according to one embodiment of the present invention. The word “vehicle” indicates a conveyance including an automobile, a railway carriage, and so on.

A plurality of the bioelectrodes 100 electrically insulated from each other is disposed in the steering wheel surfacing member. For instance, with a right hand of the driver touching any one of the bioelectrodes 100 and a left hand of the driver touching any another one of the bioelectrodes 100, those bioelectrodes 100 are electrically conducted to each other through a body of the driver serving as an electrically conducting path, and the cardiac potential as an example of the biometric information can be measured. The individual bioelectrodes 100 are connected to wirings (not illustrated), and the wirings are connected to a control device (not illustrated) to which the detected cardiac potential is input.

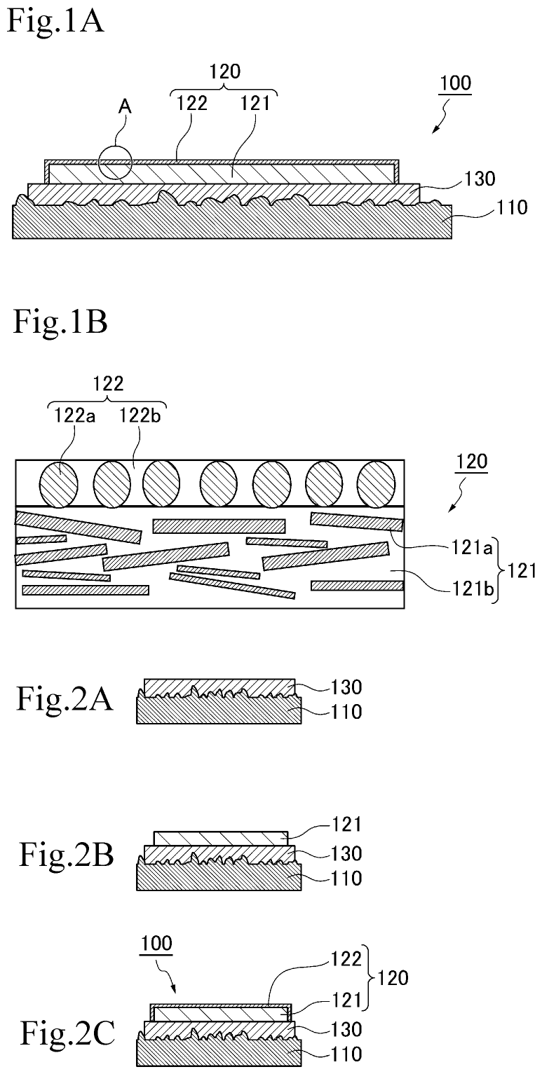

As illustrated in FIG. 1(A), the bioelectrode 100 is constituted by disposing an electrode member 120 on a surface side of a base 110 with an underlayer 130 disposed therebetween. The electrode member 120 is composed of a plurality of conductive layers laminated one above another. In more detail, the electrode member 120 has a two-layer structure in which a surface of a first conductive layer 121 is covered by a second conductive layer 122. The second conductive layer 122 covers the entirety of the first conductive layer 121 and is disposed to be exposed at a surface side of the bioelectrode 100.

The base 110 is a member on which the electrode member 120 is disposed, and it is made of leather (synthetic leather), foam, fabric, a rubber sheet, or any other suitable material. The base 110 is formed as a sheet made of any of those materials. Among the above-mentioned materials, the leather (synthetic leather) is preferably used for the base 110 from the viewpoint of texture and tactile feel. In more detail, it is preferable to use, for example, synthetic leather in which a surfacing member made of urethane resin or vinyl resin and a foam made of PP foam, urethane foam, silicone foam, or the like are laminated one above the other.

In this embodiment, the bioelectrode 100 is formed on, for example, a steering wheel or a vehicle interior member of which surface is mainly made of a material such as leather (synthetic leather), fabric, or the like. Therefore, the base 110 is a “uneven base” with irregularities on its surface, and the irregularities on the surface provide specific tactile feel and texture. In other words, the base 110 of the bioelectrode 100 is a “decorative surfacing member” capable of presenting an external appearance surface of the steering wheel or the vehicle interior component by itself. The decorative surfacing member has pliability and can be attached to the steering wheel or the vehicle interior component while it is deformed following the shape of the steering wheel or the vehicle interior component. Moreover, in this embodiment, the base 110 capable of being attached to a surface side of the steering wheel has extensibility allowing extension of the base 110.

The first conductive layer 121 is laminated on the surface side of the base 110 with the underlayer 130 interposed therebetween and functions as a main conductive layer. In this embodiment, the first conductive layer 121 is a layer that is formed by dispersing scale-shaped conductive particles 121a in an insulating binder 121b and by curing the insulating binder 121b. In an example, silver ink containing scale-shaped filler constituted as the conductive particles 121a made of scale-shaped silver, for example, can be used for the first conductive layer 121. The first conductive layer 121 formed by using that type of silver ink has low resistance and high conductivity.

The first conductive layer 121 has extensibility allowing extension thereof like the base 110. Crosslinked rubber or a thermoplastic elastomer can be generally used as a matrix forming the insulating binder 121b with extensibility. Practical examples of the crosslinked rubber may include silicone rubber, natural rubber, isoprene rubber, butadiene rubber, acrylonitrile butadiene rubber, 1,2-polybutadiene, styrene-butadiene rubber, chloroprene rubber, nitrile rubber, butyl rubber, ethylene-propylene rubber, chlorosulphonated polyethylene rubber, acrylic rubber, epichlorohydrin rubber, fluorocarbon rubber, and urethane rubber. Practical examples of the thermoplastic elastomer may include a styrene thermoplastic elastomer, an olefin thermoplastic elastomer, an ester thermoplastic elastomer, a urethane thermoplastic elastomer, an amide thermoplastic elastomer, a vinyl chloride thermoplastic elastomer, and a fluorocarbon thermoplastic elastomer. Among the above-mentioned materials, the silicone rubber is particularly preferable material because it can form the pliable first conductive layer 121 with extensibility and has relatively high durability.

The hardness of the matrix forming the insulating binder 121b is preferably in a range of 5 to 80 in terms of the A hardness stipulated in JIS K6253. If the A hardness is less than 5, the matrix is too pliable, and a problem may occur in durability of the first conductive layer 121. On the other hand, if the A hardness is more than 80, the matrix is too hard, and the first conductive layer 121 is hardly extensible and is not suitable for applications where the first conductive layer 121 is to be extended and contracted.

Conductive powder made of, for example, carbon or metal can be used for the conductive filler that is constituted as the conductive particles 121a. Particularly, metal powder with low resistance is desirably used. Among various types of metal powder, silver powder with a very low resistance value is particularly preferable. Furthermore, it is particularly preferable for the conductive filler to have a scale-like shape from the viewpoint of not only obtaining low resistance with a relatively small amount of the filled conductive filler, but also reducing change in resistivity when the first conductive layer 121 is extended. In more detail, the content of the scale-shaped powder included in the conductive particles 121a is preferably 30 to 100% by volume and more preferably 50% by volume or more and less than 100% by volume. In this embodiment, the first conductive layer 121 may contain a small amount of powder other than the scale-shaped powder to reduce the resistance value more easily than in the case of using the scale-shaped powder alone. The wording “scale-shaped powder” used here indicates a plate-like powder with an aspect ratio (major axis/thickness) of more than 2, including powder in the shape of the so-called flake or a thin piece.

The above-mentioned conductive filler is preferably mixed into the first conductive layer 121 in amount of 15 to 50% by volume. If the mixed amount is less than 15% by volume, there is a possibility that the resistance value becomes too high. If the mixed amount is more than 50% by volume, a percentage of the matrix retaining the conductive filler becomes too small, thus increasing a possibility of the occurrence of, for example, cracking in the conductive layer and hence disconnection when the conductive layer is extended.

The first conductive layer 121 is preferably formed using a liquid conductive paste by printing. A liquid composition containing the insulating binder 121b (matrix) and the conductive particles 121a (conductive filler) can be used as the liquid conductive paste. Practical examples of the liquid composition may include a combination of the conductive filler with alkenyl group-containing polyorganosiloxane and hydrogen organopolysiloxane each being curable liquid resin, a combination of the conductive filler with polyurethane polyol and isocyanate, and a mixture prepared by dispersing the conductive filler into a solution that is obtained by dissolving various types of rubbers and elastomers into a solvent. Dispersibility of the conductive filler, coatability to the base surface, and viscosity can be adjusted by adding a solvent to the conductive paste.

In the case of using the conductive paste to form the first conductive layer 121 by printing, because the conductive particles 121a have the scale-like shape, a thixotropy of the conductive paste can be adjusted to 3 to 30, and conductivity in a predetermined range of the resistance value can be obtained after solidification. Furthermore, by adjusting the thixotropy of the conductive paste forming the first conductive layer 121 to 3 to 30, patterning of the first conductive layer 121 can be performed on a surface of the base 110 with high quality. The thixotropy can be measured by using a viscometer (BROOKFIELD rotation viscometer DV-E) equipped with a spindle SC4-14 as a rotor and can be represented by a ratio (μ_{10 rpm}/μ_{100 rpm}) of a measurement value μ_{10 rpm} at a rotation speed of 10 rpm to a measurement value μ_{100 rpm} at a rotation speed of 100 rpm.

The first conductive layer 121 or the conductive paste may contain various additives for the purpose of improving various properties such as productivity, weather resistance, and heat resistance. The additives may be, for example, various function improvers such as a plasticizer, a reinforcement, a colorant, a heat resistance improver, a flame retardant, a catalyst, a curing retardant, and an anti-degradation agent.

The elastic modulus E′2 of the first conductive layer 121 is preferably 2 to 60 MPa. This is because the first conductive layer 121 itself needs to be somewhat pliable. If the elastic modulus E′2 is lower than 2 MPa, a relative amount of the conductive particles 121a contained in the first conductive layer 121 becomes too small, thus causing a possibility that the conductivity of the bioelectrode 100 cannot be obtained at a sufficient level after being extended. If the elastic modulus E′2 is higher than 60 MPa, the first conductive layer 121 becomes too hard, and wrinkles or undulations tend to remain in the base 110 made of fabric, for example. The term “elastic modulus E′” used in the Description and Claims indicates the storage modulus E′ when a specimen is pulled by a dynamic viscoelasticity measuring device in a tensile mode. The elastic modulus E′ of the first conductive layer 121 is denoted by “E′2” in some cases to distinguish it from the elastic modulus E′ of one or more other layers, for example, the underlayer. The elastic modulus E′ of the first conductive layer 121 can be measured by forming a material composition of the first conductive layer 121 into the shape of a specimen for which the elastic modulus E′ can be measured.

Because the first conductive layer 121 is formed of an extensible silver paste, it has extensibility. In more detail, the first conductive layer 121 preferably has extensibility to such an extent that the first conductive layer 121 can be extended about 30% when the bioelectrode 100 is attached to the steering wheel and about 10% after being attached. Moreover, because the first conductive layer 121 has extensibility, the thickness of the first conductive layer 121 is preferably relatively thick in terms of the aspect ratio of the first conductive layer 121 from the viewpoint of reducing change in resistance value when the first conductive layer 121 is extended. However, if the thickness of the first conductive layer 121 exceeds 100 μm, the first conductive layer 121 tends to crack. Accordingly, the thickness of the first conductive layer 121 is preferably set to be 100 μm or less in consideration of durability.

The second conductive layer 122 is laminated to cover a surface side of the first conductive layer 121 and functions as a “conductive protection layer” for compensating for low wear of the first conductive layer 121. The second conductive layer 122 is preferably formed as a coating film with such extensibility as enabling the second conductive layer 122 to follow extension of the first conductive layer 121.

In this embodiment, the second conductive layer 122 is formed by dispersing conductive particles 122a having mainly a lump shape (e.g., a spherical, elliptical, or indeterminate shape) into an insulating binder 122b such as a urethane binder or a silicone binder. In other words, the lump-shaped conductive particles 122a are mainly contained in the second conductive layer 122, and the scale-shaped conductive particles are not contained at all or almost not contained therein. The aspect ratio of the lump-shaped conductive particles 122a is preferably 2 or less. The insulating binder 122b (matrix) of the second conductive layer 122 can be made of the same material as that of the insulating binder 121b (matrix) of the above-mentioned first conductive layer 121.

Carbon ink containing carbon filler is used for the second conductive layer 122. Accordingly, the second conductive layer 122 is harder than the first conductive layer 121 and serves as a conductive layer with wear resistance. Thus, the hardness of the matrix forming the insulating binder 122b is preferably in a range of 5 to 80 in terms of the A hardness stipulated in JIS K6253. If the A hardness is less than 5, the matrix is too pliable, and a problem may occur in wear resistance and durability of the second conductive layer 122. On the other hand, if the A hardness is more than 80, the matrix is too hard, and the second conductive layer 122 is hardly extensible and is not suitable for applications where the second conductive layer 122 is to be extended and contracted.

The second conductive layer 122 can contain a larger amount of conductive particles in comparison with the first conductive layer 121. The reason is that, because the lump-shaped conductive particles are less likely to fall out than the scale-shaped conductive particles, the durability can be increased even when the relatively large amount of conductive particles are contained in the conductive layer. On that occasion, the conductive filler contained in the second conductive layer 122 can be mixed at an occupancy of 10 to 60% by volume in the second conductive layer 122. If the conductive filler is less than 10% by volume, there is a possibility that the resistance value of the second conductive layer 122 becomes too high. If the conductive filler is more than 60% by volume, a percentage of the matrix retaining the conductive filler becomes too small, thus increasing a possibility of the occurrence of, for example, cracking in the second conductive layer 122 when the second conductive layer 122 is extended.

On the other hand, from the viewpoint of further increasing the durability, the amount of the conductive particles filled in the second conductive layer 122 is preferably smaller than that filled in the first conductive layer 121. Thus, because the amount of the conductive particles filled in the second conductive layer 122 is smaller than that filled in the first conductive layer 121, the second conductive layer 122 is a conductive layer with relatively low conductivity. In more detail, the conductive filler contained in the second conductive layer 122 is preferably mixed at an occupancy of 10 to 30% by volume in the second conductive layer 122.

However, the second conductive layer 122 is not limited to a layer including the conductive particles. In other words, the second conductive layer 122 may be made of a conductive polymer such as PEDOT:PSS (poly(3,4-ethylenedioxythiophene)/poly(4-styrene sulfonate). When the conductive polymer is used for the second conductive layer 122, the conductive polymer may contain the conductive particles. The content of the conductive particles may be, for example, the same as that in the above-described case of using the insulating binder 122b, but it is preferably smaller than the latter. In more detail, the content is preferably more than 0% by volume and less than 25% by volume.

To reduce the resistance value and to ensure the conductivity, the thickness of the second conductive layer 122 is preferably 70 μm or less and more preferably 50 μm or less. Moreover, in this embodiment, because the second conductive layer 122 is disposed to be exposed at the surface side of the base 110 (namely, at the same side as an external appearing surface of the steering wheel or the vehicle interior component), the second conductive layer 122 preferably has the same or similar hue or tone as or to that of the base 110 from the viewpoint of presenting, for example, a more satisfactory appearance design of the base 110. Thus, when the second conductive layer 122 is to be black, it can be formed in black by using the carbon filler as the conductive particles 122a. With the second conductive layer 122 having the same or similar hue or tone as or to that of the base 110, the second conductive layer 122 can be made less conspicuous relative to the base 110. When there are no conductive particles in desired color, the hue or the tone can be adjusted by adding a coloring pigment or dye within a range not significantly reducing the conductivity.

The underlayer 130 is disposed between the base 110 and the first conductive layer 121 for easier coating of the first conductive layer 121 over the surface of the base 110. However, when the underlayer 130 permeates into the inside of the base 110 as in the case of the base 110 being made of natural leather, synthetic leather, or fabric, the term “underlayer 130” indicates a layer including a permeated portion as well. Furthermore, the underlayer 130 is formed of a coating film with such extensibility as enabling the underlayer 130 to follow extension of the base 110. Thus, the underlayer 130 is made of a polymer matrix and is preferably formed of the same type of material as the polymer matrix forming the first conductive layer 121 from the viewpoint of increasing adhesivity between the underlayer 130 and the first conductive layer 121. For example, insulating resin, including urethane resin such as polyurethane resin or silicone resin such as liquid silicone rubber, is used for the underlayer 130.

Even when the underlayer 130 permeates into the inside of the base 110 as described above, the underlayer 130 is formed along the surface of the base 110. Accordingly, by forming the underlayer 130 with use of the same matrix as that of the first conductive layer 121, the first conductive layer 121 and the underlayer 130 are integrated into an inseparable state, and adhesion between both the layers can be maintained even when they are extended. On that occasion, there is a possibility that the first conductive layer 121 and the underlayer 130 merge with each other and a boundary between both the layers cannot be discerned. In such a case, an upper portion with conductivity is the first conductive layer 121, and a lower portion with no conductivity is the underlayer 130.

The underlayer 130 can be formed on the base 110 at least under such a condition of occupying a larger area than the first conductive layer 121. Step-like level differences caused by the thicknesses of the underlayer 130 and the first conductive layer 121 are avoided from overlapping each other by using, as the underlayer 130, a material with higher extensibility than that of the first conductive layer 121 and by forming the underlayer 130 to extend up to a region around the first conductive layer 121. As a result, generation of a large step can be avoided.

In addition, the second conductive layer 122 is also formed to completely cover the first conductive layer 121. In other words, the second conductive layer 122 is formed in a larger area than the first conductive layer 121. With such an arrangement, since the first conductive layer 121 is surrounded by the underlayer 130 and the second conductive layer 122, the first conductive layer 121 can be protected against, for example, ingress of moisture. The protection against the ingress of moisture is important on the surface side coming into touch with the living body. However, when a material having affinity with moisture, such as a sweat absorbing material, is used for the base 110, it is also important that the underlayer 130 suppresses the ingress of moisture from the base side.

The elastic modulus E′ of the underlayer 130 (denoted by “E′1” in some cases to distinguish it from the elastic modulus of the one or more other layers) is preferably 1 to 10 MPa. This is because the underlayer 130 needs to be pliable to absorb local variations in extension of the base 110. In the case of the base 110 being made of fabric, for example, a gap between twisted yarns may locally spread when the fabric is extended. Moreover, a plurality of ridges may be formed in the base 110 depending on a method of weaving or knitting the fabric. When the fabric in such a state is extended, a gap between the adjacent ridges may locally widen. If the local widening of the gap occurs, it is important to prevent the first conductive layer 121 from being affected by the local widening of the gap. The elastic modulus E′ of the underlayer 130 is specified to avoid the above-mentioned adverse effect. If the elastic modulus E′ of the underlayer 130 is lower than 1 MPa or higher than 10 MPa, wrinkles or undulations tend to remain in the base 110. Measurement of the elastic modulus E′ of the underlayer 130 and fabrication of a specimen for the measurement can be performed in similar manners to those in the case of the first conductive layer 121

In this embodiment, the base 110 on which the electrode member 120 is disposed is the “uneven base” with irregularities in its surface. By forming the underlayer 130 such that the surface of the base 110 becomes a nearly flat surface, it is easier to perform the coating of the first conductive layer 121 or later-described transfer thereof. Furthermore, when adhesion force of the first conductive layer 121 to the base 110 is weak, the underlayer 130 can provide an effect of increasing the adhesion force. Alternatively, the first conductive layer 121 may be directly disposed on the surface of the base 110 without interposition of the underlayer 130 therebetween.

Method of Manufacturing Bioelectrode:

In this embodiment, the bioelectrode 100 is manufactured by directly laminating the underlayer 130 and then the first conductive layer 121 and the second conductive layer 122 of the electrode member 120 on the base 110, namely the “uneven base”, by printing. In more detail, as illustrated in FIG. 2(A), the underlayer 130 is first formed by coating insulating resin, for example, urethane resin such as polyurethane resin or silicone resin such as liquid silicone rubber, on the uneven surface of the base 110 by screen printing, for example. Then, as illustrated in FIG. 2(B), the first conductive layer 121 is formed by coating silver ink on a surface side of the underlayer 130 by screen printing, for example. Then, as illustrated in FIG. 2(C), the second conductive layer 122 is formed by coating carbon ink to cover the surface of the first conductive layer 121 by screen printing, for example. As a result, the bioelectrode 100 is manufactured.

The method of manufacturing the bioelectrode 100 is not limited to the above-mentioned example of directly laminating the underlayer 130 and then the first conductive layer 121 and the second conductive layer 122 of the electrode member 120 on the base 110, namely the “uneven base”, by printing, and the bioelectrode 100 can also be manufactured by any other suitable method. In an example, the manufacturing method may include the steps of forming an underlayer on each of an on-release-film print coated member and a synthetic leather base and bonding them to each other for transfer of the electrode member.

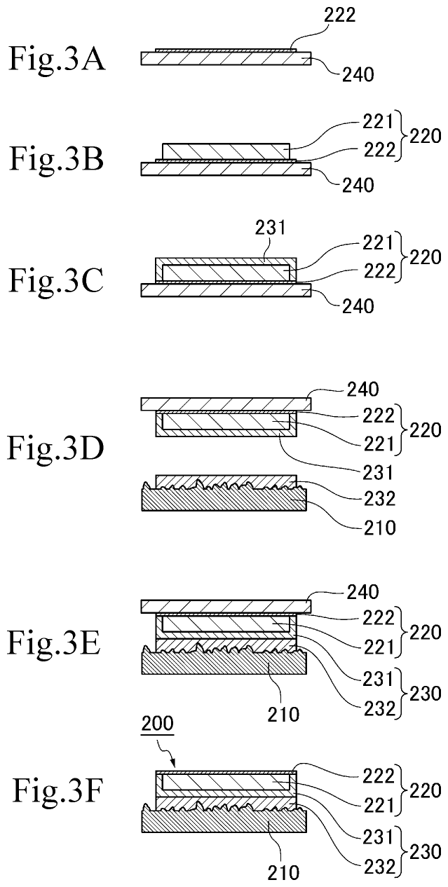

In more detail, as illustrated in FIG. 3(A), a second conductive layer 222 is formed by coating carbon ink on a surface of a release film 240 made of a silicone release PET film by screen printing, for example. Then, as illustrated in FIG. 3(B), a first conductive layer 221 is formed by coating silver ink on a surface side of the second conductive layer 222. On that occasion, the first conductive layer 221 is formed to have a smaller external contour than the second conductive layer 222 such that the second conductive layer 222 has the larger external contour than the first conductive layer 221. Thus, an electrode member 220 composed of the first conductive layer 221 and the second conductive layer 222 is formed on the release film 240. Then, as illustrated in FIG. 3(C), an underlayer 231 is formed by coating insulating resin, for example, urethane resin such as polyurethane resin or silicone resin such as liquid silicone rubber, on a surface of the first conductive layer 221 by screen printing, for example.

Then, as illustrated in FIG. 3(D), the on-release-film print coated member including the electrode member 220 and the underlayer 231 both formed on the release film 240 is positioned to face a synthetic leather base that is obtained by forming an underlayer 232 on a surface of a base 210. Then, as illustrated in FIG. 3(E), the underlayer 232 on the synthetic leather base and the underlayer 231 on the on-release-film print coated member are bonded to each other. Then, as illustrated in FIG. 3(F), the release film 240 is peeled off. As a result, a bioelectrode 200 is formed in which the electrode member 220 is laminated on the surface side of the base 210 with an underlayer 230 interposed therebetween.

The method of manufacturing the bioelectrode by forming the underlayer on each of the on-release-film print coated member and the synthetic leather base and bonding them to each other is not limited to the above-mentioned example, and the bioelectrode can also be manufactured by another suitable method. In an example, the manufacturing method may include the steps of coating underlayer ink on an on-release-film print coated member, bonding the on-release-film print coated member to a synthetic leather base, and curing the underlayer ink.

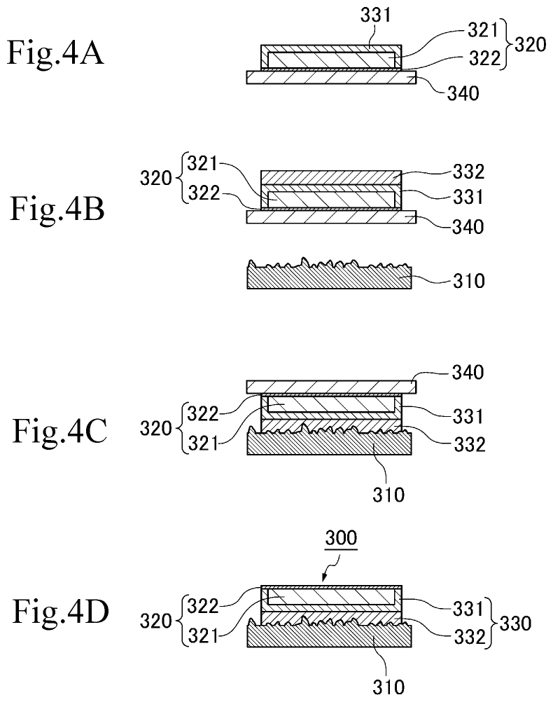

In more detail, as illustrated in FIG. 4(A), a second conductive layer 322 is formed by coating carbon ink on a surface of a release film 340 made of a silicone release PET film by screen printing, for example. Then, a first conductive layer 321 is formed by coating silver ink on a surface side of the second conductive layer 322 by screen printing, for example. Then, an underlayer 331 is formed by coating insulating resin, for example, urethane resin such as polyurethane resin or silicone resin such as liquid silicone rubber, on a surface of the first conductive layer 321 by screen printing, for example.

Thereafter, as illustrated in FIG. 4(B), underlayer ink 332 is coated on an upper surface side of the underlayer 331 on the on-release-film print coated member. Then, as illustrated in FIG. 4(C), a synthetic leather base 310 and the underlayer 332 on the on-release-film print coated member are bonded to each other. Then, as illustrated in FIG. 4(D), by thermally curing the underlayer ink 332, a bioelectrode 300 is formed in which an electrode member 320 is laminated on a surface side of the base 310 with an underlayer 330 interposed therebetween.

Advantageous Effects of Embodiment:

According to this embodiment, in the bioelectrode 100, the electrode member 120 with a two-layer structure of the first conductive layer 121 and the second conductive layer 122 each having extensibility is disposed on part of the surface of the base 110. Therefore, accuracy in detecting the biometric information, such as the cardiac potential of the driver grasping the steering wheel, in the form of an electrical signal can be increased without impairing the tactile feel and the texture of the base 110 as far as possible. In addition, by designing the bioelectrode 100 to have the same or similar hue or tone as or to that of the base 110, an external appearance is hardly impaired, and more satisfactory texture can be obtained.

According to this embodiment, the electrode member 120 disposed on the surface side of the base 110 of the bioelectrode 100 has the two-layer structure of the first conductive layer 121 with low resistance and high conductivity and the second conductive layer 122 containing a smaller amount of the filled conductive particles than the first conductive layer 121 and being harder than the first conductive layer 121. Particularly, since the first conductive layer 121 functioning as the main conductive layer includes the scale-shaped filler that is constituted as the scale-shaped conductive particles 121a dispersed in the insulating binder 121b, the first conductive layer 121 can have the low resistance and the high conductivity, but the scale-shaped filler is apt to fall out. In consideration of that point, according to this embodiment, the second conductive layer 122 is coated on the surface side of the first conductive layer 121. As a result, the conductive particles 121a in the first conductive layer 121 can be easily retained and the falling-out of the conductive particles 121a can be reduced.

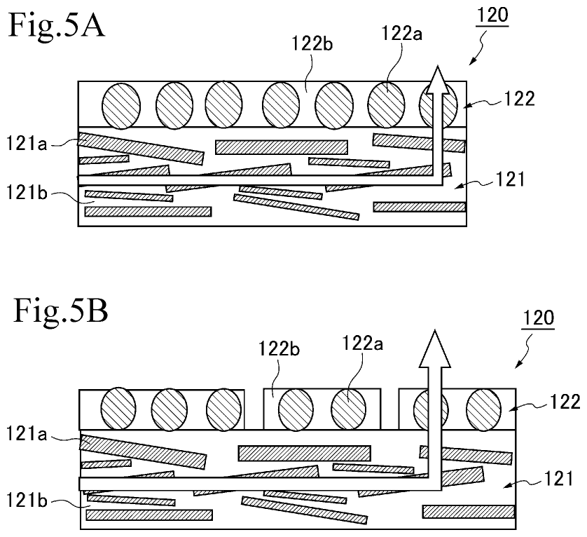

Furthermore, the conductive particles 122a contained in the second conductive layer 122 have the lump shape, and the second conductive layer 122 includes no scale-shaped conductive particles or includes a trace amount. Thus, the scale-shaped filler is contained in the first conductive layer 121 functioning as the main conductive layer to increase the conductivity and the extensibility. On the other hand, no scale-shaped filler is contained in the second conductive layer 122 functioning as the “conductive protection layer” for the first conductive layer 121. Therefore, the electrode member 120 of the bioelectrode 100 can be constituted in the laminated structure capable of suppressing the falling-out of the conductive particles while high conductivity and extensibility are ensured. Stated in another way, as illustrated in FIG. 5(A), the first conductive layer 121 functioning as the main conductive layer can maintain high conductivity in a planar direction (lengthwise direction) because of including the scale-shaped filler, while conductivity of the first conductive layer 121 in a lamination direction (thickness direction) can be ensured by coating the second conductive layer 122 with conductivity on the surface side of the first conductive layer 121.

According to this embodiment, the first conductive layer 121 is a pliable conductive layer with extensibility, but the second conductive layer 122 is relatively hard conductive layer in comparison with the first conductive layer 121. Therefore, the first conductive layer 121 can maintain the conductivity on condition that it does not rupture when extended. On the other hand, the second conductive layer 122 has properties tending to rupture when extended, but it also has durability enough for serving as the protection layer for the first conductive layer 121 because the second conductive layer 122 is the relatively hard conductive layer. Thus, as illustrated in FIG. 5(B), even when the second conductive layer 122 is ruptured in some places with extension of the first conductive layer 121 of the electrode member 120, the first conductive layer 121 and the second conductive layer 122 are kept firmly fixed to each other without peeling off. As a result, the conductivity of the first conductive layer 121 in the lamination direction (thickness direction) can be ensured with the second conductive layer 122 fixed to the first conductive layer 121, while the high conductivity is ensured in the planar direction (lengthwise direction) of the first conductive layer 121.

In the case of using a conductive polymer, such as PEDOT/PSS, to form the second conductive layer 122, the conductive polymer is not at a sufficient level from the viewpoint of toughness in the form of a coating film, but it contains no conductive particles or contains a trace amount. When that type of conductive polymer is laminated on the first conductive layer 121, the falling-out of the conductive particles 121a contained in the first conductive layer 121 can be suppressed by the laminated conductive polymer. Moreover, although the conductive polymer has lower conductivity than the silver paste, the lower conductivity of the conductive polymer is not problematic for the reason as follows. When the conductive polymer is used as the second conductive layer 122, the second conductive layer 122 simply needs to have conductivity in the thickness direction at a place where a human body touches the second conductive layer 122.

In the bioelectrode 100 according to this embodiment, as described above, since the second conductive layer 122 of the electrode member 120 is exposed to the outside, the electrode member 120 is directly touched by the hand. Thus, the second conductive layer 122 on an outer side of the electrode member 120 of the two-layer structure is required to have higher durability. From that point of view, the second conductive layer 122 is made of a harder material.

According to this embodiment, when the bioelectrode 100 in the flat sheet shape is attached to a rim portion surface of a doughnut-shaped steering wheel such that the bioelectrode-equipped steering-wheel surfacing member is held in close contact with the rim portion surface, the bioelectrode 100 is extended while curving along the rim portion. In consideration of the above point, the first conductive layer 121 serving as the main conductive layer of the electrode member 120 is given with a certain level of extensibility allowing the first conductive layer 121 to extend about 30% at the time of attachment and about 10% after the attachment. Therefore, the bioelectrode-equipped steering-wheel surfacing member including the bioelectrode 100 can be attached in close contact with the shape of the steering wheel. In addition, since the high conductivity of the first conductive layer 121 is not impaired even when the bioelectrode-equipped steering-wheel surfacing member is attached in close contact with the steering wheel, the detection accuracy of the bioelectrode 100 can be increased.

Description of Cardiac Potential Measuring System:

Outline of a cardiac potential measuring system using the bioelectrode 100 according to the embodiment of the present invention will be described below with reference to the drawings.

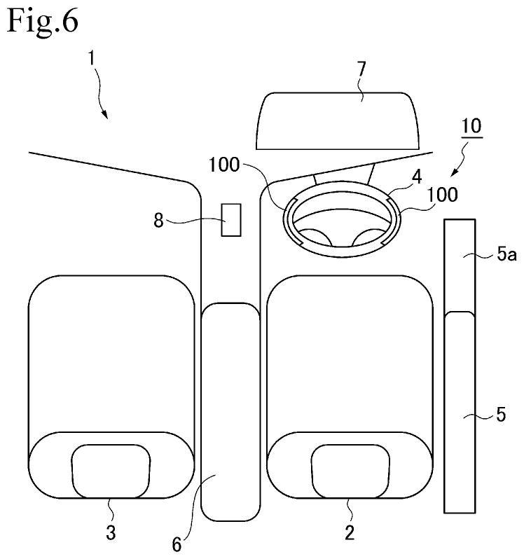

The bioelectrode 100 according to the embodiment functions as an electrocardiogram sensor and can be applied to a cardiac potential measuring system 10 for detecting, in the form of an electrical signal, a cardiac potential as an example of the biometric information of a driver of an automobile 1 that is an example of the “vehicle”. As illustrated in FIG. 6, for example, in the automobile 1 including not only a driver seat 2, a passenger seat 3, and a steering wheel 4 that is an example of the “steering device”, but also a door-side armrest 5 on a door inner panel 5a, a center console-side armrest 6, an instrument panel 7, and a shift lever 8 that are each an example of the “vehicle interior component”, the cardiac potential measuring system 10 is constituted by disposing the bioelectrode 100 in at least a rim-portion surfacing member of the steering wheel 4.

In more detail, a plurality of the bioelectrodes 100 is disposed on the steering wheel 4 of the automobile 1 to be capable of being touched by the right and left hands of the driver, and the bioelectrodes 100 function as an electrocardiogram sensor for detecting variations in a myocardial action potential with the heartbeats of the driver while the driver is grasping the steering wheel 4 by both the hands. Accordingly, the biometric information, such as the cardiac potential, of the driver grasping the steering wheel 4 can be detected through the bioelectrodes 100 with high accuracy. The cardiac potential measuring system 10 using the bioelectrodes 100 according to the embodiment may also be applied to other vehicles, such as a railway carriage, than the automobile 1.

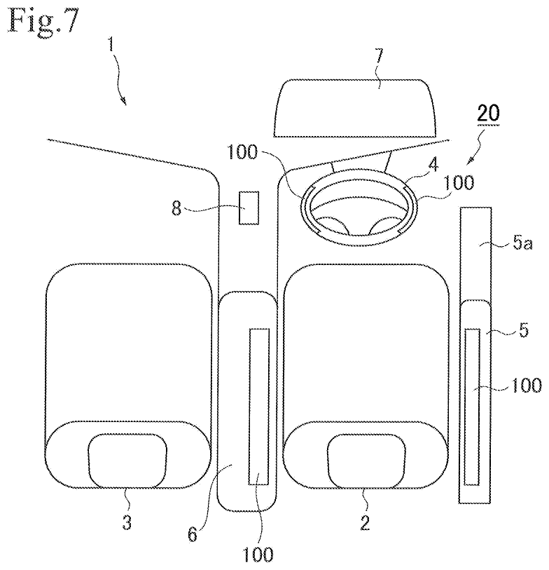

As illustrated in FIG. 7, a cardiac potential measuring system 20 may be constituted by disposing the bioelectrode 100 according to the embodiment in each of surfacing members for the door-side armrest 5 and the center console-side armrest 6 on both sides of the driver seat 2 in addition to the steering wheel 4 of the automobile 1. Alternatively, the bioelectrode 100 may be disposed in either one of the door-side armrest 5 and the center console-side armrest 6.

With the above-described configuration of the cardiac potential measuring system 20, the bioelectrodes 100 function as an electrocardiogram sensor, for example, when the driver grasps the steering wheel 4 by the right hand and touches the center console-side armrest 6 by the left hand, or when the driver grasps the steering wheel 4 by the left hand and touches the door-side armrest 5 by the right hand. Accordingly, it is possible to know the biometric information, such as the cardiac potential, of the driver in a similar manner to that in the above-described embodiment. The door-side armrest 5 and the center console-side armrest 6 each including the bioelectrode 100 are examples of the “bioelectrode-equipped vehicle interior component” according to the embodiment of the present invention.

In the cardiac potential measuring system 20 according to this embodiment, the bioelectrode 100 simply needs to be disposed on, in addition to the steering wheel 4, a surface side of at least one of vehicle components, such as the door inner panel 5a including the door-side armrest 5, the center console-side armrest 6, and the shift lever 8, which are each positioned within a range reachable by the driver's hand. The cardiac potential measuring system 20 using the bioelectrodes 100 according to this embodiment may also be applied to other vehicles, such as a railway carriage, than the automobile 1.

While the embodiments of the present invention have been described in detail above, it will be easily understood by those skilled in the art that the present invention can be variously modified insofar as not substantially departing from the novel matters and the advantageous effects of the present invention. Thus, all those modifications also fall within the scope of the present invention.

For instance, the terms used at least once in the Description or the drawings together with different terms that are broader than or equivalent to the former terms can be replaced with the different terms regardless of where the former terms are used in the Description or the drawings. Moreover, the configuration and the operation of the bioelectrode and the cardiac potential measuring system are not limited to those described in the embodiments of the present invention and can be variously modified.

### REFERENCE SIGNS LIST

4 steering wheel, 5 door-side armrest (vehicle interior component), 5a door inner panel (vehicle interior component), 6 center console-side armrest (vehicle interior component), 7 instrument panel (vehicle interior component), 8 shift lever (vehicle interior component), 10, 20 cardiac potential measuring system, and 100, 200, 300 bioelectrode

1. A bioelectrode capable of detecting biometric information of a living body in touch with the bioelectrode, the bioelectrode comprising:
a base;
a first conductive layer that is laminated on a surface side of the base, that is formed by dispersing scale-shaped conductive particles in an insulating binder, and that has extensibility; and
a second conductive layer that is laminated on a surface side of the first conductive layer, that has conductivity, and that is harder than the first conductive layer,
wherein the second conductive layer is disposed to be exposed at the surface side of the base where the second conductive layer is touchable with the living body.

2. The bioelectrode according to claim 1,
wherein the second conductive layer includes lump-shaped conductive particles, and an amount of the conductive particles filled in the second conductive layer is smaller than an amount of the conductive particles filled in the first conductive layer.

3. The bioelectrode according to claim 1,
wherein the second conductive layer is made of a conductive polymer.

4. The bioelectrode according to claim 1,
wherein the second conductive layer has a larger outer contour than the first conductive layer.

5. The bioelectrode according to claim 1,
wherein the second conductive layer is disposed to cover a surface of the first conductive layer.

6. The bioelectrode according to claim 1,
wherein the second conductive layer has same color tone as the base.

7. The bioelectrode according to claim 1,
wherein a thickness of the first conductive layer is at least 100 μm or less, and a thickness of the second conductive layer is at least 70 μm or less.

8. The bioelectrode according to claim 1,
further comprising an insulating underlayer between the base and the first conductive layer.

9. A bioelectrode-equipped steering-wheel surfacing member comprising one or more bioelectrodes according to claim 1, the one or more bioelectrodes being disposed on a steering wheel surfacing member.

10. A bioelectrode-equipped vehicle interior component comprising one or more bioelectrodes according to claim 1, the one or more bioelectrodes being disposed on a vehicle interior component.

11. A cardiac potential measuring system for detecting, as biometric information of a driver driving a vehicle, a cardiac potential in form of an electrical signal, the cardiac potential measuring system comprising:
a plurality of the bioelectrodes according to claim 1,
the bioelectrodes including:
a first bioelectrode disposed on a steering device of the vehicle, the steering device being operated by the driver; and
a second bioelectrode disposed on the steering device or a vehicle interior component disposed in a compartment of the vehicle.

12. The cardiac potential measuring system according to claim 11, wherein the vehicle interior component is at least one of a door inner panel, a center console-side armrest, or a shift lever.

13. The bioelectrode according to claim 1,
wherein the base is an uneven base with irregularities on its surface.

14. The bioelectrode according to claim 1,
wherein a conductive filler that is constituted as the conductive particles is mixed into the first conductive layer in amount of 15 to 50% by volume.

15. The bioelectrode according to claim 1,
wherein a conductive filler contained in the second conductive layer is mixed at an occupancy of 10 to 60% by volume in the second conductive layer.

16. The bioelectrode according to claim 8,
wherein the underlayer is formed on the base at least under such a condition of occupying a larger area than the first conductive layer.

17. The bioelectrode according to claim 1,
wherein the base has extensibility allowing extension capable of presenting an external appearance surface of the steering wheel or the vehicle interior component by itself,
wherein the first conductive layer has extensibility allowing extension thereof like the base.

18. The bioelectrode according to claim 1,
wherein the second conductive layer is formed as a coating film with such extensibility as enabling the second conductive layer to follow extension of the first conductive layer.

19. The bioelectrode according to claim 8,
wherein the underlayer is formed of a coating film with such extensibility as enabling the underlayer to follow extension of the base.

20. The bioelectrode according to claim 19,
wherein a material of the underlayer has higher extensibility than that of the first conductive layer.
